SEQUENCE LISTING (1 sequence)

SEQ ID NO: 1 - protein, 585 aa, Artificial Sequence
  human serum albumin
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

(19) United States
(12) Patent Application Publication
(10) Pub. No.: US 2015/0165000 A1
(43) Pub. Date: Jun. 18, 2015

(54) COMPOSITION FOR THERMOSTABILIZATION OF HUMAN SERUM ALBUMIN AND METHOD OF PREPARING THERMALLY STABILIZED HUMAN SERUM ALBUMIN USING THE SAME

(71) Applicants: Dong Hae Shin, Seoul (KR); Gil Ja Jhon, Seoul (KR); Mi Sun Kim, Seoul (KR); Ji Min Park, Busan (KR)

(72) Inventors: Dong Hae Shin, Seoul (KR); Gil Ja Jhon, Seoul (KR); Mi Sun Kim, Seoul (KR); Ji Min Park, Busan (KR)

(73) Assignee: Ewha University - Industry Collaboration Foundation, Seoul (KR)

(21) Appl. No.: 14/357,995

(22) PCT Filed: Dec. 18, 2013
(86) PCT No.: PCT/KR2013/011792

§ 371 Date: May 13, 2014

(30) Foreign Application Priority Data
Dec. 18, 2012 (KR) 10-2012-0148271
Feb. 4, 2013 (KR) 10-2013-0012592
Aug. 6, 2013 (KR) 10-2013-0093289

(51) Int. Cl. A61K 38/38; C07K 14/765; A61K 47/12

(52) U.S. Cl. CPC A61K 38/385; A61K 47/12; C07K 14/765

(57) ABSTRACT

The present invention relates to a composition for thermostabilization in the preparation of a human serum albumin formulation, comprising a fatty acid selected from the group consisting of C16 to C22 fatty acids or a pharmaceutically acceptable salt thereof; a method of preparing thermally stabilized human serum albumin, comprising: 1) adjusting a concentration of human serum albumin to 1 to 40 mg/mL; and 2) adding the fatty acid or pharmaceutically acceptable salt thereof to the human serum albumin; and a method of preparing a human serum albumin formulation, comprising adding the fatty acid or pharmaceutically acceptable salt thereof to human serum albumin and thermally treating the resultant.

### TECHNICAL FIELD

The present invention relates to a composition for thermostabilization in the preparation of a human serum albumin formulation, comprising a fatty acid selected from the group consisting of C16 to C22 fatty acids or a pharmaceutically acceptable salt thereof; a method of preparing thermally stabilized human serum albumin, comprising: 1) adjusting a concentration of human serum albumin to 1 to 40 mg/mLmL; and 2) adding the fatty acid or pharmaceutically acceptable salt thereof to the human serum albumin; and a method of preparing a human serum albumin formulation, comprising adding the fatty acid or pharmaceutically acceptable salt thereof to human serum albumin and thermally treating the resultant.

Background Art

Albumin is a protein which is very abundant in the blood and is made in the liver. Structurally, human serum albumin (HSA) is composed of 585 amino acids (66,438 Da), 17 disulfide bridges and one free cysteine (Cys34) [Dugiaczyk, A. at al., Proc. Natl Acad. Sci. USA, 1982, 79: 71-75]. Furthermore, albumin functions to maintain and restore the volume of plasma, and is coupled with a variety of ligands such as water, calcium, sodium and potassium cations, fatty acids, hormones, bilirubin, drugs, etc. to adjust the colloid osmotic pressure of blood and to deliver the ligand, and plays a role as an amino acid source in the case of poor nutrition [Bar-Or, D. at al., Eur J. Biochem., 2001, 268: 42-47]. In particular, the binding of drug and albumin is greatly associated with expression of drug efficacy. Meanwhile, purified HSA is used to treat hypoalbuminemia caused due to albumin loss and albumin synthesis dysfunction, including surgical operations, hemorrhagic shock or burns and nephrotic syndromes. Albumin is used as a drug additive for supplementing a medium for use in the growth of higher eukaryotic cells and for mixing a therapeutic protein. Also, serum albumin is supposed to act as an oxygen carrier which is adsorbed to or desorbed from oxygen depending on the partial pressure of oxygen, like hemoglobin. Hence, in emergency cases, albumin may be administered in lieu of hemoglobin. Currently, the demand for albumin products is appropriated by albumin extracted from human blood.

Conventional HSA is prepared by subjecting human plasma to the low-temperature ethanol fractionation method of Cohn or any method similar thereto to give an HSA-containing fraction (fraction V) and then performing a variety of purification methods. These purification methods may include purification methods typically used in protein chemistry, for example, a salting out method, ultrafiltration, isoelectric point precipitation, electrophoresis, ion exchange chromatography, gel permeation chromatography, affinity chromatography, etc. Because samples obtained from plasma actually contain many kinds of contaminants derived from biotissue, cells, blood and so on, human serum albumin needs to be purified using combinations of the above methods. However, the amount of the purified albumin from the human plasma is inevitably limited. Accordingly, methods of preparing human serum albumin have been recently developed using yeast [Ken, O. et al., J. Biochem., 1991, 110: 103-110] and Bacillus subtilis [Saunders, C. W. et al., J. Bacteriol., 1987, 169: 2917-2925] by use of genetic engineering without having to depend on human plasma as a raw material.

Typically, albumin formulations in a form of an aqueous solution undergo a process of a thermal treatment at 60° C. for 10 hr in order to inactivate viruses capable of contaminating the formulations. The industrial production of albumin is executed under conditions different from the environment in the human body. When considering the properties of albumin, which is easily denatured and aggregated by heat, etc., albumin may be easily denatured or may be readily formed into multimers in the course of a series of procedures. Currently commercially available 5% or 20% albumin formulations are widely utilized without particular side effects and the multimers contained in the formulations are thus considered to be harmless to the human body, but side effects such as increase in blood pressure, fever, tachycardia, tremor, flushing, urticaria, chills, etc. are reported by use of denatured albumin injections. Hence, to prevent denaturation due to thermal treatment, currently commercially available albumin formulations are prepared by adding sodium octanoate and N-acetyl tryptophan as a stabilizer.

Research into the various useful functions of albumin is ongoing. Taking into consideration the high possibility of development of novel albumin formulations, the demand thereof is expected to continuously increase. However, because the single supply source of human serum albumin is currently the blood of humans and is thus limited to some extent, development of a novel supply source is urgently required, as well as research into the properties of albumin. In consideration of the current advancement of technology in life science technique fields, recombinant human serum albumin using a transformant will be able to play a role as another supply source for the production of albumin formulations.

Meanwhile, as mentioned above, albumin is coupled with various materials, such as water, ions, fatty acids, hormones, etc., and thus exhibits physiological activity. Therefore, in order to utilize recombinant human serum albumin in the production of an albumin formulation, there is a need to identify an element essential for the physiological activity of albumin among these materials, investigate how it is coupled, and apply it to recombinant human serum albumin, thereby rendering the recombinant human serum albumin physiologically active.

### DISCLOSURE

### Technical Problem

Culminating in the present invention, intensive and thorough research into identification of ligands naturally bound to physiologically active human serum albumin in vivo and into discovery of materials able to stabilize albumin upon thermal treatment, which is an essential process for the preparation of albumin formulations, was carried out by the present inventors aiming to solve the problems encountered in the related art, and resulted in the finding that a C16 to C22 saturated or unsaturated fatty acid such as oleic acid, linoleic acid, etc. may be bound to human serum albumin, and the fatty acid may improve thermal stability of albumin, thus preventing denaturation in the process of a thermal treatment necessary for production of albumin formulations, and furthermore, when the fatty acid or salt thereof is added to human serum albumin having a concentration of 1 to 40 mg/mLmL, high stability may be maintained even upon thermal treatment for a long period of time of about 10 hr.

### Technical Solution

An object of the present invention is to provide a composition for thermostabilization in preparing a human serum albumin formulation, comprising a fatty acid selected from the group consisting of C16 to C22 fatty acids or a pharmaceutically acceptable salt thereof.

Another object of the present invention is to provide a method of preparing a thermally stabilized human serum albumin, comprising: 1) adjusting a concentration of human serum albumin to 1 to 40 mg/mLmL; and 2) adding a fatty acid selected from the group consisting of C16 to C22 fatty acids or a pharmaceutically acceptable salt thereof to the human serum albumin.

A further object of the present invention is to provide a method of preparing a human serum albumin formulation, comprising adding a fatty acid selected from the group consisting of C16 to C22 fatty acids or a pharmaceutically acceptable salt thereof to human serum albumin and thermally treating the resultant.

### Advantageous Effects

According to the present invention, when a fatty acid selected from the group consisting of C16 to C22 fatty acids is added, it can be efficient at producing albumin that is stable even in the course of thermal treatment in preparation of a human serum albumin formulation. In particular, even when human serum albumin having a concentration of 1 to 40 mg/mLmL, added with the fatty acid, is thermally treated at a high temperature of 60° C. or more for 10 hr or longer, human serum albumin maintained in stability can be prepared. On the other hand, in the case of using recombinant human serum albumin, recombinant human serum albumin which is considerably similar to human-derived albumin in terms of structure can be produced, and thus can be industrially used without side effects. Even in the case of using plasma-derived human serum albumin, because a composition including various fatty acids naturally present in vivo instead of a foreign material is employed, a desired formulation can be produced without the worry of toxicity or side effects.

### DESCRIPTION OF DRAWINGS

FIG. 1 illustrates a three dimensional (3D) structure of human serum albumin crystals obtained from a 20% human serum albumin injection by Green Cross Corp. made of plasma-derived human serum albumin commercially available in the form of a formulation, as obtained by an X-ray crystallography method, wherein the binding positions of octanoic acid and L-tryptophan which is a decay product of N-acetyl tryptophan used as thermal stabilizers in the conventional preparation process of plasma-derived human serum albumin are represented by stick models, and the binding position of oleic acid as a C18 fatty acid is represented by a sphere model;

FIG. 2 illustrates results of mass analysis of a 20% human serum albumin injection by Green Cross Corp. made of plasma-derived human serum albumin commercially available in the form of a formulation;

FIG. 3 illustrates results of mass analysis of a 20% human serum albumin injection by SK made of plasma-derived human serum albumin commercially available in the form of a formulation;

FIG. 4 illustrates the endurable time duration of the prolonged processing depending on temperatures represented by a minute unit and a log unit thereof in a fatty acid-free human serum albumin solution and an oleic acid-containing human serum albumin solution;

FIG. 5 illustrates results of measurement of circular dichroism of five human serum albumin samples (Green Cross albumin, A3782 albumin, A1653 albumin, Green Cross albumin+oleic acid (1:1 molar ratio), A3782 albumin+oleic acid (1:1 molar ratio)) when heated at 60° C. for 10 hr;

FIG. 6 illustrates results of measurement of circular dichroism of five human serum albumin samples (Green Cross albumin, A3782 albumin, A1653 albumin, Green Cross albumin+oleic acid (1:1 molar ratio), A3782 albumin+oleic acid (1:1 molar ratio)) when heated at 70° C. for 10 hr;

FIG. 7 illustrates results of measurement of circular dichroism of five human serum albumin samples (Green Cross albumin, A3782 albumin, A1653 albumin, Green Cross albumin+oleic acid (1:1 molar ratio), A3782 albumin+oleic acid (1:1 molar ratio)) when heated at 80° C. for 10 hr;

FIG. 8 illustrates results of measurement of circular dichroism of ten human serum albumin samples having different concentrations (1 to 45 mg/mLmL A3782 albumin+oleic acid (1:1 molar ratio)) when heated at 70° C. for 10 hr; and

FIG. 9 illustrates results of measurement of circular dichroism of five human serum albumin samples having different fatty acids (A3782 albumin+oleic acid (1:1 molar ratio), A3782 albumin+linoleic acid (1:1 molar ratio), A3782 albumin+α-linolenic acid (1:1 molar ratio), A3782 albumin+γ-linolenic acid (1:1 molar ratio), A3782 albumin+palmitoleic acid (1:1 molar ratio)) when heated at 70° C. for 10 hr.

### BEST MODE

In order to accomplish the above objects, an aspect of the present invention provides a composition for thermostabilization in preparing a human serum albumin formulation, comprising a fatty acid selected from the group consisting of C16 to C22 fatty acids or a pharmaceutically acceptable salt thereof.

Use of recombinant human serum albumin in the method of preparing the human serum albumin formulation according to the present invention makes it possible to solve conventional problems in which the supply source for production of a plasma-derived albumin formulation is limited. However, human serum albumin is coupled with a variety of ligands in vivo to form a stable structure and show physiological activity, and thus, in order to formulate recombinant human serum albumin, the structure of physiologically active albumin and/or ligands bound thereto should be taken into consideration first.

Meanwhile, in conventional production of a formulation from plasma-derived human serum albumin, foreign materials such as sodium octanoate and N-acetyl tryptophan, the stability of which has not been verified, are added to impart stability against thermal treatment, but there is a need to discover materials naturally present in vivo instead thereof. Therefore, the present inventors have ascertained that a fatty acid selected from the group consisting of C16 to C22 fatty acids is bound to plasma-derived human serum albumin, and thus such a fatty acid may stabilize the human serum albumin, may especially impart stability against thermal treatment, and thereby, by adding to recombinant human serum albumin a fatty acid selected from the group consisting of C16 to C22 fatty acids, albumin that is stable even in the course of thermal treatment may be formed, making it possible to produce formulations using the same.

As used herein, the term “albumin” refers to a protein which constitutes a basic material of cells, is very abundantly present in the blood and is produced in the liver. Among simple proteins present in nature, albumin has the lowest molecular weight. Serum albumin in blood functions to maintain and restore the volume of plasma and is thus used to prevent shock due to massive hemorrhage and is useful for operation and burn treatment. Furthermore, albumin is supposed to have oxygen transport capability similar to hemoglobin. In the present invention, the composition of the present invention is responsible for imparting stability against thermal treatment in the course of formulating the albumin, and the target albumin may include all albumins which may be formulated, and preferably includes human serum albumin derived from human plasma or recombinant human serum albumin made by genetic engineering.

As used herein, the term “recombinant human serum albumin” is a term used as a concept which is in contrast with natural human serum albumin derived from human plasma, and commonly designates artificially produced human serum albumin. For example, it may be human serum albumin obtained from a transformant which is prepared to express a polypeptide having an amino acid sequence of SEQ ID NO: 1 (sequence of HSA), and may have the same amino acid sequence as in the human serum albumin isolated from the plasma, but is not limited thereto.

As used herein, the term “transformant” refers to a genetically modified organism which expresses a specific gene via genetic engineering. In the present invention, the specific gene may preferably be a gene for encoding a polypeptide having an amino acid sequence of SEQ ID NO: 1, for example, human serum albumin. Preferable examples of the genetically modified organism may include, but are not limited to, cells such as transformed bacteria, fungi and yeast, or transgenic animals. Preferable examples of the transgenic animals may include, but are not limited to, mammals, such as mice, rats, guinea pigs, etc.

As used herein, the term “human serum albumin formulation” refers to a concept including all formulations made of human serum albumin, which may be formulated in the form of being administrable to a subject including human, and may include commercially available albumin formulations, and formulations prepared to replace them, having components, functions and efficacies similar to the commercially available albumin formulations. In the case of commercially available albumin formulations, they have problems in that they depend on the blood as a limited supply source. As an alternative thereto, recombinant human serum albumin formulations may be prepared using recombinant human serum albumin. Preferably, the recombinant human serum albumin formulation according to the present invention may contain, as an effective component, recombinant human serum albumin obtained from a transformant prepared to express human serum albumin of SEQ ID NO: 1.

In order for the human serum albumin to be prepared in the form of a formulation which may be administered to the human body, it is necessary for a process of a thermal treatment to prevent virus contamination and/or other infections. However, human serum albumin, like typical proteins, may be structurally changed or agglutinated and thus denatured upon treatment at high temperature. Hence, an additive is required to stabilize the albumin. Currently, in the preparation process of plasma-derived albumin formulations, an octanoic acid, for example, sodium octanoate and N-acetyl tryptophan is being utilized. However, these materials are not originally present in plasma but are optionally added to improve stability in the process of a thermal treatment, and may thus cause side effects when administered to the human body, unlike natural human serum albumin.

Accordingly, upon preparation of human plasma-derived or recombinant human serum albumin formulations, a shape and structure which is the most similar to those of natural human serum albumin need to be maintained as much as possible. Furthermore, because recombinant human serum albumin is different in terms of some properties from human serum albumin extracted from human plasma, the case where a composition for thermostabilization in the preparation of recombinant human serum albumin formulations contains a material existing in vivo in place of the optional additive may be safer. Therefore, a C16 to C22 fatty acid identified as bound to human serum albumin isolated from plasma is used in the present invention.

As used herein, the term “C16 to C22 fatty acid” gives a general name to a fatty acid having 16 to 22 carbon atoms among fatty acids, and is configured to include a hydrophilic carboxyl group at one end thereof and a hydrophobic carbon chain at the other end thereof. As such, the kind of fatty acid may vary depending on the number and position of double bonds in the carbon chain, and is preferably selected from palmitic acid and palmitoleic acid as C16 fatty acids, stearic acid (octadecanoic acid), oleic acid, linoleic acid and linolenic acid as C18 fatty acids, eicosapentaenoic acid (EPA) as a C20 fatty acid, docosahexaenoic acid (DHA) as a C22 fatty acid, and combinations thereof, but is not limited thereto. Moreover, the fatty acid may be used in the form of a pharmaceutically acceptable salt.

As used herein, the term “palmitic acid” refers to a saturated fatty acid having a 16-carbon chain, and is hexadecanoic acid in IUPAC nomenclature. It has a chemical formula of CH₃(CH₂)₁₄CO₂H, and is the most typical fatty acid found in plants and animals, and microorganisms. It is also present as the main component of palm oil, or exists in meat, cheese, butter and dairy products. The salt and ester of palmitic acid are referred to as palmitate. At basic pH, it is present in the form of a palmitate anion. Excessive carbohydrates in vivo are converted into palmitic acid. Palmitic acid is the first fatty acid produced in the synthesis process of fatty acid, and a precursor of long-chain fatty acid. Some proteins may be biologically coupled with a palmitoyl group, and thus modified, which is called palmitoylation that is important for membrane localization of protein.

As used herein, the term “palmitoleic acid” refers to omega-7 monounsaturated fatty acid, cis-palmitoleic acid or 9-cis-hexadecenoic acid, and is hexadec-9-enoic acid in IUPAC nomenclature. It is a C16 fatty acid with a single double bond, has a chemical formula of CH₃(CH₂)₅CH═CH(CH₂)₇CO₂H and is a constituent of glyceride in human adipose tissue. It is present in all tissues, and is mainly found at higher concentration in the liver. It is bio-synthesized from palmitic acid by the action of delta-9 desaturase. Palmitoleic acid is beneficial and inhibits destruction of insulin-secreting pancreatic beta cells and suppresses inflammation to thus increase insulin sensitivity. Edible palmitoleic acid may be extracted from oils of animal/plant and marine life.

As used herein, the term “stearic acid” refers to a saturated fatty acid having an 18-carbon chain and is octadecanoic acid in IUPAC nomenclature. It is a waxy solid with a chemical formula of CH₃(CH₂)₁₆CO₂H. The salt and ester thereof are named as stearate. Stearic acid is one of the saturated fatty acids which are most widely present in nature, together with palmitic acid. Because it has a polar head portion linkable with a metal ion and a non-polar chain which provides solubility in an organic solvent, it has double functionality. Owing to such properties, it may be used as a surfactant or a softener.

As used herein, the term “oleic acid” refers to colorless odorless oil produced from oils and fats of various plants and animals. Commercially available products may be yellowish. It is chemically classified as monounsaturated omega-9 fatty acid, and has a chemical formula of CH₃(CH₂)₇CH═CH(CH₂)₇CO₂H. Also, oleic acid is the most abundant in human adipose tissue. As used herein, the term “linoleic acid” refers to an unsaturated n-6 fatty acid and is a colorless liquid at room temperature. This linoleic acid is one of essential fatty acids which cannot be synthesized from other food components in vivo. In particular, it is a polyunsaturated fatty acid used for biosynthesis of arakidonic acid and also some prostaglandin. It is found in membrane lipids and abundant in vegetable oils. It is a carboxylic acid having a 18-carbon chain and two cis double bonds in the chemical structure, wherein the first double bond is located at the sixth carbon from a methyl end which is hydrophobic, and it has a chemical formula of CH₃(CH₂)₄CH═CHCH₂CH═CH(CH₂)₇COOH.

As used herein, the term “linolenic acid” generically refers to two types of α- and γ-linolenic acids or a mixture thereof. Linolenic acid is present in vegetable oils in the form of ester (linolenate). In particular, α-linolenic acid is a carboxylic acid having a 18-carbon chain and three cis double bonds, and is a polyunsaturated n-3 fatty acid wherein the first double bond is located at the third carbon from a methyl end, namely, n-end, and is chemically called all-cis-9,12,15-octadecatrienoic acid. As the isomer thereof, γ-linolenic acid has a chain of the same number of carbon atoms with the same number of double bonds as in α-linolenic acid, but is a polyunsaturated n-6 fatty acid wherein the first double bond is located at the sixth carbon from n-end, and has different positions of double bonds in the chain. γ-Linolenic acid is also called gamolenic acid, and is known to be effective against inflammatory and autoimmune diseases.

As used herein, the term “EPA (eicosapentaenoic acid)” refers to omega-3 fatty acid, called timnodonic acid, and is a carboxylic acid having a 20-carbon chain and five cis double bonds in the chemical structure. The first double bond is located at the third carbon from the omega end, that is, the hydrocarbon end. EPA is a polyunsaturated fatty acid (PUFA), and acts as a precursor for eicosanoids, such as, prostaglandin-3 (which inhibits platelet aggregation), thromboxane-3 and leukotriene-5. EPA is present in a large amount in seaweed and fish, especially fish oils, and also in maternal milk of human. Fish cannot naturally produce EPA but obtain it from algae which they consume. Although α-linolenic acid may be converted into EPA in the human body, the efficiency thereof is very low. Meanwhile, EPA acts as a precursor to DHA. EPA has a chemical formula of CH₃CH₂ (CH═CHCH₂)₅ (CH₂)₂COOH.

As used herein, the term “DHA (docosahexaenoic acid)” refers to omega-3 fatty acid which is a primary component of the human brain, cerebral cortex, skin, sperm, testicles and retina. It may be synthesized from α-linolenic acid or may be directly obtained from maternal milk or fish oils. It is a carboxylic acid having a 22-carbon chain and six cis double bonds in the chemical structure. The first double bond is located at the third carbon from the omega end, that is, the hydrocarbon end. DHA is cervonic acid in trivial name, and is (4Z,7Z,10Z,13Z,16Z,19Z)-docosa-4,7,10,13,16,19-hexaenoic acid in IUPAC nomenclature. Deep-sea fish oils are known to be rich in DHA. DHA originates from photosynthetic and heterotrophic microalgae and becomes increasingly concentrated in organisms the further they are up the food chain. DHA may be produced via conversion of α-linolenic acid in vivo, and the conversion rate is higher by about 15% in women. Upon administration of testosterone or suppression of conversion from testosterone into estradiol, conversion into DHA is reduced. DHA is a major fatty acid in sperm, brain phospholipids and retina, and dietary DHA may reduce the risk of heart disease by reducing the level of blood triglycerides. Moreover, below-normal levels of DHA are associated with Alzheimer's disease.

These fatty acids may be represented by N:X(n-Y) depending on the number of lipids, wherein N is the number of carbon atoms in the entire chain, that is, 16, 18, 20 or 22, X is the number of double bonds contained in the chain, and Y designates the position of carbon at which the double bond starts from the methyl end of each fatty acid. As such, ω may be used in lieu of n. For example, in the present invention, palmitic acid is a C16 saturated fatty acid and is thus represented by 16:0, palmitoleic acid has a single double bond at the seventh carbon from the methyl end and is thus represented by 16:1(n-7) or 16:1(ω-7), stearic acid is a C18 saturated fatty acid and is thus represented by 18:0, oleic acid has a single double bond at the ninth carbon from the methyl end and is thus represented by 18:1(n-9) or 18:1(ω-9), linoleic acid is represented by 18:2(n-6) or 18:2(ω-6), α-linolenic acid is represented by 18:3(n-3) or 18:3(ω-3), γ-linolenic acid is represented by 18:3(n-6) or 18:3(ω-6), EPA is represented by 20:5(n-3), and DHA is represented by 22:6(n-3).

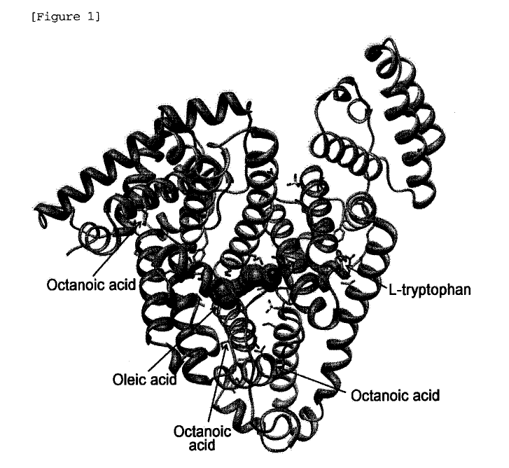

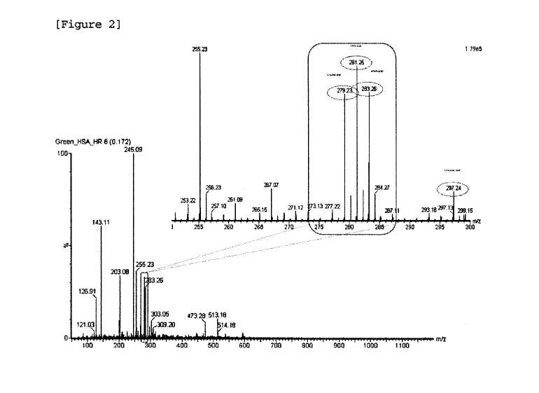

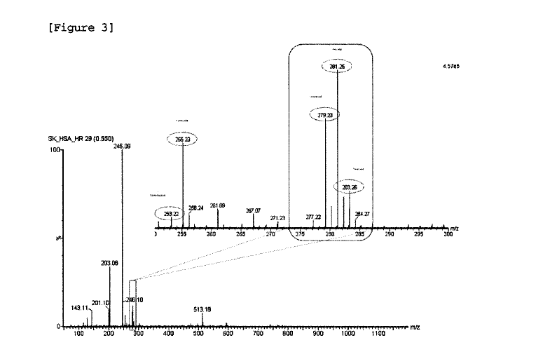

In an embodiment of the present invention, as is shown by results of measurement of the structure of an injection made of plasma-derived human serum albumin and of mass analysis thereof, binding of C18 fatty acid including oleic acid was confirmed (FIGS. 2 and 3). Also, the position of the fatty acid bound to albumin was determined (FIG. 1). Thus, oleic acid or another C18 fatty acid, C16 fatty acid, EPA or DHA was added to a fatty acid-free human serum albumin solution, followed by performing high-temperature treatment, whereby human serum albumin added with C18 oleic acid was not denatured and was maintained stably for a much longer period of time (two times as long or more) even at a high temperature of 70° C., compared to the fatty acid-free human serum albumin solution without oleic acid (Tables 3 to 6), and the albumin solution containing the other C18 fatty acid was sustained without deformation for a longer period of time compared to the fatty acid-free albumin solution (Table 7). In addition, when using C16 fatty acid, EPA and DHA, the same effects were exhibited (Tables 8 and 9).

As used herein, the term “pharmaceutically acceptable salt” refers to all salts which retain desired biological and/or physiological activities of the composition, without toxicity to cells or human exposed to the composition, in which undesired toxicological effects are minimized. Used as the salt is an acid addition salt formed by a pharmaceutically acceptable free acid. The acid addition salt is prepared using a typical method, including, for example, dissolving a compound in an excess of acid aqueous solution, and precipitating the resulting salt using a water-miscible organic solvent, for example, methanol, ethanol, acetone or acetonitrile. The equimolar compound and the acid or alcohol (e.g. glycol monomethyl ether) in water are heated, and the mixture is evaporated and thus dried, or the precipitated salt may be suction filtered. As such, the free acid may include inorganic acid and organic acid, and examples of the inorganic acid may include hydrochloric acid, hydrobromic acid, phosphoric acid, nitric acid, sulfuric acid, tartaric acid, etc., and examples of the organic acid may include, but are not limited to, methane sulfonic acid, p-toluene sulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc.

Also, a pharmaceutically acceptable metal salt is preferably prepared using a base. An alkali metal or alkaline earth metal salt is obtained by, for example, dissolving a compound in an excess of alkali metal hydroxide or alkaline earth metal hydroxide solution, filtering the insoluble compound salt, and evaporating and drying the filtrate. As such, a proper example of the metal salt is a sodium salt, a potassium salt or a calcium salt from the pharmaceutical point of view, but is not limited thereto. Furthermore, a silver salt corresponding thereto may result from reacting an alkali metal or alkaline earth metal salt with an appropriate silver salt (e.g. silver nitrate).

Unless otherwise stated, the C16 to C22 fatty acid or pharmaceutically acceptable salt thereof includes salts of acidic or basic groups which may be present in C16 to C22 fatty acids. For example, pharmaceutically acceptable salts may include sodium, calcium and potassium salts of hydroxyl group, and the other pharmaceutically acceptable salts of amino group may include hydrobromide, sulfate, hydrogen sulfate, phosphate, hydrogen phosphate, dihydrogen phosphate, acetate, succinate, citrate, tartrate, lactate, mandelate, methanesulfonate (mesylate) and p-toluenesulfonate (tosylate), and may be prepared using a salt preparation method known in the art. Preferable examples thereof may include, but are not limited to, alkali metal or alkaline earth metal salts.

Preferably, the composition for thermostabilization according to the present invention may be added that the fatty acid or pharmaceutically acceptable salt thereof becomes at a molar ratio of 1:0.5 to 1:2, preferably 1:0.7 to 1:1.5, more preferably 1:0.8 to 1:1.2, and much more preferably 1:1, relative to the albumin, but the present invention is not limited thereto.

Another aspect of the present invention provides a method of preparing thermally stabilized human serum albumin, comprising: 1) adjusting a concentration of human serum albumin to 1 to 40 mg/mLmL; and 2) adding a fatty acid selected from the group consisting of C16 to C22 fatty acids or pharmaceutically acceptable salt thereof to the human serum albumin.

The C16 to C22 fatty acid or the pharmaceutically acceptable salt thereof, the preferable adding amount thereof and the human serum albumin are as described above.

With the goal of overcoming the problem of side effects of infectious diseases mediated by albumin reported in the past, thermal treatment at 60° C. for 10 hr was introduced along with virus killing effects using cold ethanol. Although albumin is a structurally very stable protein, it may be formed into dimers, oligomers and polymers during the above thermal treatment, and in severe cases, gelation is reported to occur. Thus, in the process of a thermal treatment necessary for preparation of human serum albumin formulations, the development of methods able to maintain stability of human serum albumin for a long period of time is regarded as important. Therefore, in the present invention, when human serum albumin having a low concentration of 1 to 40 mg/mLmL was processed with a C16 to C22 fatty acid or pharmaceutically acceptable salt thereof, the secondary structure of albumin was proven to be maintained even upon thermal treatment at 60 to 80° C. for 10 hr or longer. The method of the present invention enables the albumin structure to be stabilized even upon thermal treatment for ones of hours, thereby ensuring availability for development of albumin formulations having increased stability.

Preferably, steps of 1) and 2) are performed sequentially or reversely. The method of preparing thermally stabilized human serum albumin according to the present invention is characterized in that the human serum albumin sample added with the C16 to C22 fatty acid may be maintained in stability upon thermal treatment for ones of hours when its concentration is adjusted to the low level of 1 to 40 mg/mLmL and then thermally treated. As such, the C16 to C22 fatty acid relative to the human serum albumin may be added at a molar ratio of 1:0.5 to 1:2, preferably 1:0.7 to 1:1.5, more preferably 1:0.8 to 1:1.2, and much more preferably 1:1. Accordingly, step 2) for adding the human serum albumin with the C16 to C22 fatty acid may be performed before or after adjusting the concentration of human serum albumin, regardless of the step sequence, so long as the final concentration of human serum albumin is maintained at the above level.

According to the preparation method of the present invention, in the case where the human serum albumin having a low concentration of 1 to 40 mg/mLmL processed with the C16 to C24 fatty acid is heated, 80 to 100% of the α-helix of the human serum albumin before heating may be maintained during up 20 to 5 to 16 hr of heating.

In order to adjust the concentration of the human serum albumin to 1 to 40 mg/mLmL, a variety of dilution buffers typical for preparation of albumin formulations may be employed. Examples of the dilution buffers may include a histidine buffer, a citrate buffer, a succinate buffer, an acetate buffer, or a phosphate buffer, and preferably used is a mixed solution of 50 mM potassium phosphate and 150 mM NaCl (pH 7.5), but the present invention is not limited thereto.

Because the thermally stabilized human serum albumin provided by the method of the present invention is intended to be formulated and administered to humans as mentioned above, the preparation method thereof may include a process of a thermal treatment for preventing contamination and/or potential infection.

To prepare the albumin formulation, the human serum albumin may be heated to 60 to 80° C., preferably 65 to 75° C., and more preferably 70° C. As such, even when the thermally stabilized human serum albumin according to the present invention is heated at the above temperature for 6 to 15 hr, preferably 8 to 12 hr, and more preferably 10 hr, the α-helix of the human serum albumin may be maintained.

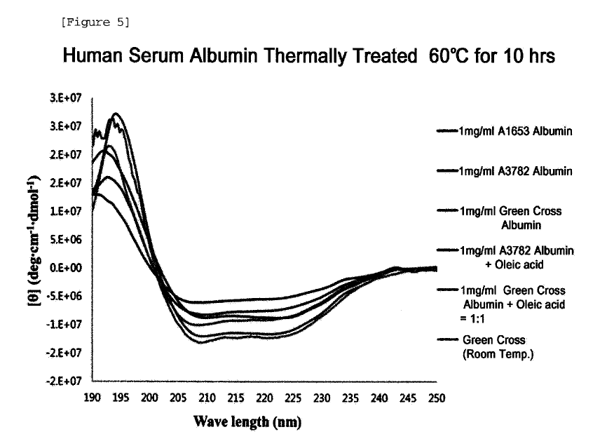

In an embodiment of the present invention, human serum albumin having a concentration of 1 mg/mLmL was processed with oleic acid at a molar ratio of 1:1 and then heated at 60 to 80° C. for 10 hr, after which the structural stability of the albumin was observed via the composition of the α-helix from the circular dichroism measurement values. As a result, stability of the albumin was maintained at 60° C. and 70° C. but was deteriorated at 80° C. or higher (Example 7, FIGS. 5 to 7).

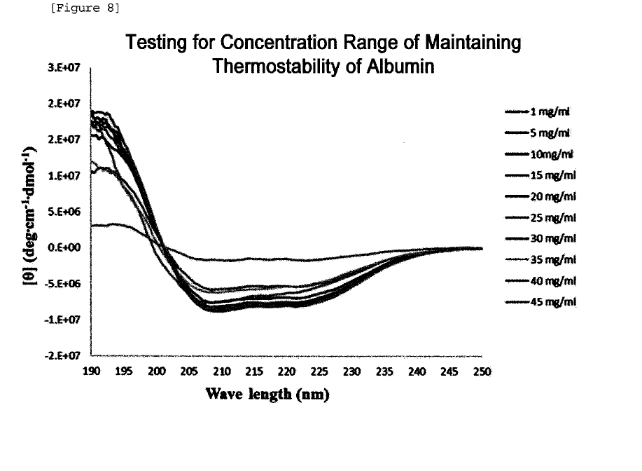

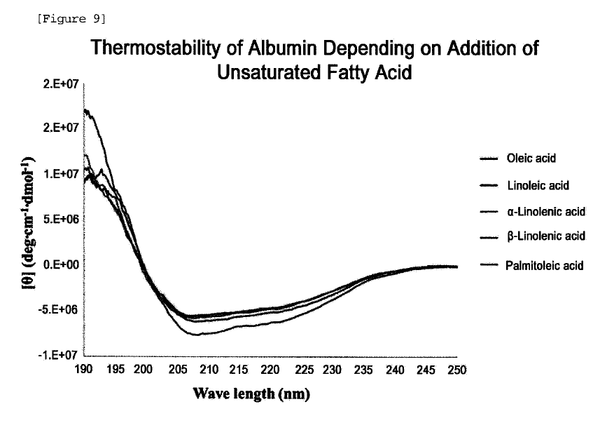

Also, even after the human serum albumin having a concentration of 1 to 40 mg/mLmL was processed with oleic acid at a molar ratio of 1:1 and then heated at 70° C. for 10 hr, stability thereof was maintained (Example 8, FIG. 8). Furthermore, stability of the albumin was maintained even after addition of fatty acids other than oleic acid such as linoleic acid, α-linolenic acid, γ-linolenic acid and palmitoleic acid and then heating at 70° C. for 10 hr (Example 9, FIG. 9).

This indicates that thermal stability of human serum albumin having a low concentration of 1 to 40 mg/mLEL is remarkably increased by the addition of the C16 to C24 fatty acid like oleic acid.

A further aspect of the present invention provides a method of preparing a human serum albumin formulation, comprising adding a fatty acid selected from the group consisting of C16 to C22 fatty acids or pharmaceutically acceptable salt thereof to human serum albumin and thermally treating the resultant.

The C16 to C22 fatty acid or the pharmaceutically acceptable salt thereof, the preferable adding amount thereof and the human serum albumin are as described above.

As used herein, the term “processing” generically refers to all of procedures for incorporating a C16 to C24 fatty acid or pharmaceutically acceptable salt thereof in any form of chemical or biological functions in order to achieve thermostabilization. For example, in a culturing procedure for preparing thermally stabilized human serum albumin, the fatty acid may be incorporated into a medium composition, or may be incorporated in the form of a composition in the course of processing of the human serum albumin. In this way, the processing may be applied at any step before thermal treatment.

Preferably, the human serum albumin formulation according to the present invention may be prepared using albumin isolated from human plasma or recombinant human serum albumin formed by genetic engineering.

The human serum albumin formulation according to the present invention is intended to be administered to human as mentioned above, and thus the preparation method thereof includes thermal treatment for preventing contamination and/or infection potential. Accordingly, to prevent protein denaturation due to the thermal treatment, processing the human serum albumin with the fatty acid selected from the group consisting of C16 to C22 fatty acids or pharmaceutically acceptable salt thereof so as to be thermally stabilized is implemented.

In a conventional process of preparing a human serum albumin formulation, stability against thermal treatment has been imparted using sodium octanoate and/or N-acetyl tryptophan. However, in the present invention, the C16 to C22 fatty acid was confirmed to have thermostabilizing effects equal or superior to the above compounds. Also, in the case of C18 fatty acid which is contained in the human serum albumin formulation, a shape and structure which are most similar to those of natural human serum albumin may be maintained without side effects.

In the preparation of the formulation according to the present invention, in the case where recombinant human serum albumin is used, it is preferably obtained from a transformant which expresses human serum albumin that is a polypeptide having an amino acid sequence of SEQ ID NO: 1. As such, the transformant is defined as above.

As used herein, the term “processing” generically refers to all of the procedures for incorporating a C16 to C22 fatty acid or pharmaceutically acceptable salt thereof in any form of chemical or biological functions in order to achieve thermostabilization. For example, in the culturing procedure for preparing human serum albumin, the fatty acid may be incorporated into a medium composition, or may be incorporated in the form of a composition in the course of processing of the human serum albumin. In this way, the processing may be applied at any step before thermal treatment.

Preferably, in the case where recombinant human serum albumin is obtained from transformed cells, the preparation method according to the present invention may include culturing the transformed cells. As such, processing with the C16 to C22 fatty acid or pharmaceutically acceptable salt thereof may be accomplished by adding the composition of the invention to a culture medium for culturing transformed cells and performing culturing.

The transformed cells include not only transformed bacteria, fungi and yeast but also cells isolated from transgenic animals which express human serum albumin.

The recombinant human serum albumin is isolated from cultured transformed cells or cells isolated from transgenic animals, cultures or culture broth of the cells, or blood or plasma of transgenic animals, and/or purified, or the human serum albumin is isolated from human plasma and/or purified, after which it may be processed with the composition for thermostabilization comprising the fatty acid or pharmaceutically acceptable salt thereof according to the present invention.

The isolated human serum albumin may be human serum albumin which is obtained from blood or serum of a transgenic animal or a transformed cell which expresses human serum albumin; from a culture or culture broth of the cell; or from blood or serum of human, and/or purified.

A method of preparing a human serum albumin formulation currently used in the art includes thermal treatment at 60° C. for 10 hr to inactivate viruses which may be contained therein, for example, AIDS and Type B hepatitis virus.

In a specific embodiment of the present invention, while a fatty acid-free human serum albumin solution having a physiologically active concentration of, for example, 50 mg/mLmL was processed with various C16 to C22 fatty acids at a molar ratio of 1:1 and thermally treated at 60° C. or higher, the extent of denaturation of protein was determined. Consequently, there was a difference in the extent of denaturation depending on the kind of fatty acid up to the temperature of about 70° C., but the denaturation rate was decreased compared to the human serum albumin solution not processed with the fatty acid. Particularly, in the case where processing with oleic acid was performed, denaturation was not observed for 10 hr or longer at 60° C., which is the thermal treatment temperature used in a general method of preparing a human serum albumin formulation, and also did not occur for about 6 hr even at a higher temperature, 65° C. and the stability was maintained (Table 3).

As mentioned above, the thermally stabilized human serum albumin may be provided by adding the C16 to C22 fatty acid before or after adjusting the concentration of the albumin, so long as the concentration of the human serum albumin is maintained to the low level of 1 to 40 mg/mLmL. In an embodiment of the present invention, the thermally stabilized human serum albumin may have stability which enables the secondary structure thereof to be maintained for 10 hr or longer even when the thermal treatment temperature is increased up to 80° C. Furthermore, denaturation does not occur for several minutes even at 100° C. but a stable state may be maintained. Thus, in the method of preparing the human serum albumin formulation according to the present invention, the human serum albumin may be adjusted to a low concentration of 1 to 40 mg/mLmL before or after processing with the C16 to C22 fatty acid or pharmaceutically acceptable salt thereof.

The case where the concentration of the albumin is adjusted to the low level as above may prolong a period of time in which denaturation does not occur and a stable state is maintained even when the temperature is increased up to 80° C. Thus, in the case where a process of a thermal treatment for a long period of time is required, the concentration of the human serum albumin may be adjusted to 1 to 40 mg/mLmL.

In the method of preparing the human serum albumin formulation according to the present invention, when thermal treatment at a high temperature of about 80° C. for a long period of time is required, the concentration of the sample containing the human serum albumin may be adjusted to 1 to 40 mg/mLmL before or after processing with the C16 to C22 fatty acid or pharmaceutically acceptable salt thereof.

In the case where the sample is adjusted to a concentration of 1 to 40 mg/mLmL, it may be heated to 60 to 80° C., which corresponds to a process of a thermal treatment for preventing contamination and/or infection potential so that the thermally stabilized human serum albumin is formulated and administered to a human as mentioned above.

After the process of a thermal treatment, concentrating to the appropriate concentration to be applied to a subject, for example, at least a physiologically active concentration, namely, 50 to 200 mg/mL, may be performed. This corresponds to concentrating the human serum albumin sample having an adjusted concentration of 1 to 40 mg/mL for thermal treatment for a long period of time, to an appropriate concentration so as to be administered to a subject including a human, after thermal treatment.

This concentrating step may be performed using a typical concentrating method, and for example, may be carried out until an appropriate concentration is obtained by means of a centrifugation method using a ultrafiltration membrane having 10 to 30 kD of MWCO (Molecular Weight Cut Off), but the present invention is not limited thereto.

The appropriate concentration of the human serum albumin formulation is not particularly limited so long as it is adapted for a human serum albumin formulation, and may be set to 50 to 200 mg/mL.

The human serum albumin formulation thus prepared may be administered to a subject including a human by means of any device able to deliver an active material to a target cell. The preferable administration mode and formulation may be provided in the form of an injection, including intravenous injection, subcutaneous injection, intradermal injection, muscular injection, drop injection, etc. The injection may be prepared using an aqueous solvent such as normal saline, Ringer's solution, etc., and a non-aqueous solvent such as vegetable oil, higher fatty acid ester (e.g. ethyl oleate, etc.), alcohol (e.g. ethanol, benzylalcohol, propyleneglycol, glycerin, etc.), and may include a pharmaceutical carrier, such as a stabilizer for preventing degeneration (e.g. ascorbic acid, sodium bisulfite, sodium pyrosulfite, BHA, tocopherol, EDTA, etc.), an emulsifier, a buffer for adjusting pH, a preservative for suppressing growth of microorganisms (e.g. phenylmercuric nitrate, thimerosal, benzalconium chloride, phenol, cresol, benzylalcohol, etc.) and so on.

### Mode for Invention

A better understanding of the present invention may be obtained through the following examples which are set forth to illustrate, but are not to be construed to limit the present invention.

### Example 1

### Search for Crystallization Conditions of Plasma-Derived Human Serum Albumin

In order to analyze the structure of plasma-derived human serum albumin having physiological activity, human serum albumin available from Green Cross Corp. was crystallized. Below, the optimal concentration for crystallization determined via precipitation testing was set to 80 mg/mL. Initial crystallization was performed using an Index Screen reagent and Screen I & II as a screening kit available from Hampton research (Naguna Niguel, Ca, USA) based on sparse matrix theory (Jancarik & Kim, 1991), by means of a manual or automatic method using a hanging drop or sitting drop vapor diffusion method. The automatic method was conducted using Hydra e-Drop (Thermo Scientific, Waltham, Mass., USA) for use in a high-throughput crystallization system manufactured in the present lab.

For crystallization adapted for subsequent data collection, 150 mg/mL was applied to determine the optimal concentration for crystallization of human serum albumin available from Green Cross Corp. through a manual refine screen.

The final crystallization conditions of the human serum albumin thus determined were as follows: a mother liquor including 35% PEG 600, 0.1 M MES (2-(N-Morpholino) ethanesulfonic Acid) pH 6.5 and 0.2 M ammonium sulfate, that is, 200 μl of a crystallization solution was placed in a well, and 2 μl of a hanging drop prepared by mixing the crystallization solution and a 150 mg/mL protein solution at a ratio of 1:1 was placed on a cover glass, followed by making crystals using a hanging drop diffusion method.

### Example 2

### Search for Cryo Conditions of Plasma-Derived Human Serum Albumin

Searching for stabilized cryo conditions is essential for the data collection process using a synchrotron. This is because the intensity of radiation from the synchrotron is strong, and thus crystals are easily decayed in the course of data collection, making it impossible to achieve complete data collection. Thus, the conditions of the crystallization solution, which enable flash freezing of crystals, should be searched.

Thus in the present invention, LV CryoOil (MiTeGen, Ithaca, N.Y.) was used for human serum albumin available from Green Cross Corp. 2 μl of LV CryoOil was placed next to the drop containing the crystals of the human serum albumin available from Green Cross Corp., and the crystals were fished out using a mounting loop and then immersed in LV CryoOil, after which the crystals were fished out using a mounting loop and then instantly frozen.

### Example 3

### X-Ray Data Collection of Plasma-Derived Human Serum Albumin

Data for human serum albumin available from Green Cross Corp. was collected up to 2.17 Å at the X-ray Australian Synchrotron. The detector was ADSC Q315r. Individual numerals are shown in Table 1 below. Also, the 3D structure of the serum-derived human serum albumin crystals was calculated therefrom. The results are shown in FIG. 1.

TABLE 1
X-ray Data Statistics for Human Serum
Albumin from Green Cross Corp.

| X-ray source | Australian Synchrotron | |
|---|---|---|
| X-ray wavelength (Å) | 0.9536 | |
| Temperature (K) | 100 | |
| Space group | C2 | |
| Unit cell parameter | | |
| a (Å) | 203.6 | |
| b (Å) | 113.5 | |
| c (Å) | 86.8 | |
| β(°) | 104.9 | |
| Volume fraction of solvent (%) | 49.99 | |
| Vₘ(Å³/Dalton) | 2.46 | |
| Resolution range (Å) | 19.78-2.17 | (2.29-2.17) |
| Unique reflections | 96299 | (11416) |
| R_{sym}^{†}(%) | 5.3 | (28.5) |
| Data completeness (%) | 96.2 | (78.3) |
| Average I/σ | 10.8 | (2.7) |

(The values in parentheses are for the highest resolution shell.)

### Example 4

### Solution of Phase Problems of Plasma-Derived Human Serum Albumin Using Molecular Replacement

The crystal structure of the human serum albumin available from Green Cross Corp. was determined by molecular replacement. Specifically, in the case where structural similarity between proteins is expected, solving phase problems by means of molecular replacement using a conventionally known structure was applied. This method determines the orientation of a molecule using a fast rotation function, and the position of a molecule via calculation of translation function, R-factor search, correlation search, etc. The approximate orientation and position thus obtained were further refined using rigid body refinement or R-factor minimization search, followed by processing refinement of atomic position and model rebuilding. In this research, solution for the human serum albumin available from Green Cross Corp. was found out by using the EPMR with the structure of apo-human serum albumin (apo-HSA, PDB ID: 1AO6) as a search model.

### Example 5

### Determination of 3D Crystal Structure of Plasma-Derived Human Serum Albumin

The phases of the human serum albumin from Green Cross Corp. were obtained via molecular replacement using a human albumin model. The initial model was built using COOT and O as graphic softwares, and energy minimization was carried out using CNS. The final model was obtained using PHENIX. The refinement parameters of the final model were summarized in Table 2 below.

TABLE 2
Data Statistics and Refinement Parameters for
Human Serum Albumin from Green Cross Corp.
Data collection statistics

| Wavelength (Å) | 0.95369 | |
|---|---|---|
| Resolution (Å) | 19.78-2.17 | |
| Redundancy | 7.1 | (6.2) |
| Unique reflections | 96299 | (11416) |
| Completeness (%) | 96.2 | (78.3) |
| I/σ | 10.8 | (2.7) |
| Rsym^{b} (%) | 5.3 | (28.5) |

parameters and refinement statistics

| | |
|---|---|
| Space group | C2 |
| Cell dimensions | 203.6 Å × 113.5 Å × 86.8 Å |
| Volume fraction of solvent | 49.99% |
| Vₘ (Å³/Dalton) | 2.46 |
| Total number of residues | 1743 |
| Total non-H atoms | 14109 |
| Number of Oleic acid | 3 |
| Number of Octanoic acid | 9 |
| Number of SO₄ | 9 |
| Number of L-Tryptophan | 3 |
| Number of water molecules | 402 |

Average temperature factors

| | | |
|---|---|---|
| Protein | 46.070 | Å² |
| Solvent | 36.518 | Å² |
| All substrate | 41.910 | Å² |
| Resolution range of reflections used | 19.78-2.17 | Å |
| Amplitude cutoff | 0.0 | σ |

| | |
|---|---|
| R-factor | 19.25% |
| Free R-factor | 24.28% |

Stereochemicalideality:

| | | |
|---|---|---|
| Bond | 0.008 | Å |

| | |
|---|---|
| Angle | 1.080* |
| Chirality | 0.073* |
| Planarity | 0.005* |
| Dihedral | 15.483* |

Ramachandran plot

| | |
|---|---|
| Residues in most favored regions | 91.8% |
| Residues in additional allowed regions | 7.7% |
| Residues in generously allowed regions | 0.6% |
| Residues in disallowed regions | 0.0% |

The 3D structure thus identified is typically similar to that of conventional apo-HSA. The binding positions of octanoic acid and N-acetyl tryptophan used in the preparation process of the pharmaceutical grade plasma-derived human serum albumin were identified. Two octanoic acids were bound at the positions known as the fatty acid binding sites, and N-acetyl tryptophan was bound to Sudlow site II. Furthermore, unexpected fatty acid binding was detected. As shown by results of analysis of X-ray structure and mass spectrometry, the above fatty acid was identified to be a C18 fatty acid such as oleic acid. The results of mass analysis of 20% human serum albumin injections currently commercially available from Green Cross Corp. and SK are shown in FIGS. 2 and 3, respectively.

### Example 6

### Effects of Fatty Acids on Thermal Denaturation of Human Serum Albumin

6.1. Effect of Oleic Acid on Thermal Denaturation of Human Serum Albumin

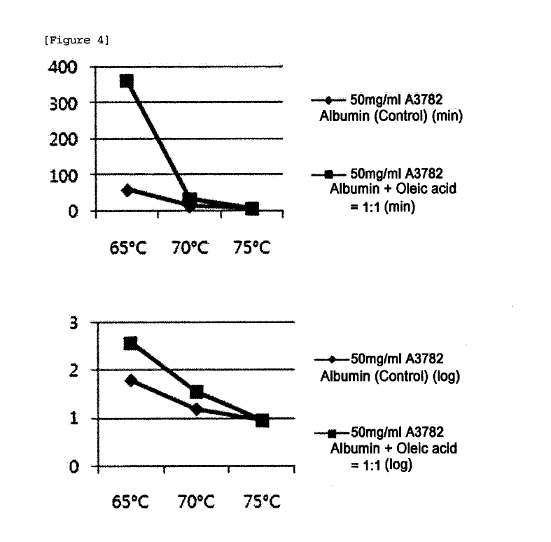

As is apparent from the results of identification of the structure of human serum albumin available from Green Cross Corp., the effect of oleic acid bound to the plasma-derived human serum albumin was evaluated. Thus, the present inventors estimated oleic acid affecting stability depending on the temperature. Accordingly, oleic acid was added to a fatty acid-free human serum albumin (A3782, Sigma-Aldrich) solution and then thermal treatment was performed at high temperature, after which the generated changes were observed. The results are shown in Tables 3 to 6 below and FIG. 4. As a control, a fatty acid-free human serum albumin solution not processed with oleic acid was used. The concentration of the human serum albumin solution was 50 mg/mL, and this solution was used in an amount of 25 μl and oleic acid was added at the same molar ratio in a test group. Specifically, while the temperature was maintained at 65° C., 70° C. and 75° C., whether the precipitation was formed due to denaturation of protein was checked at intervals of 30 min, 5 min and 3 min, respectively. At the highest temperature of 75° C., there was no difference between the control and the test group. However, in the control, precipitation began to form after 10 min at 70° C. and after 30 min at 65° C., whereas in the test group containing oleic acid, precipitation began to form after 30 min at 70° C. and after 330 min at 65° C. Therefore, oleic acid was confirmed to greatly contribute to stabilization of human serum albumin at high temperature. Specifically, because the addition of oleic acid may impart stability against thermal treatment which is essential to form a formulation, oleic acid may be used instead of sodium octanoate and N-acetyl tryptophan which have been used as conventional thermal stabilizers.

TABLE 3
65° C. Thermal treatment Conditions

| | Min | | | | | | | | | | | |
|---|---|---|---|---|---|---|---|---|---|---|---|---|
| Protein | 30 | 60 | 90 | 120 | 150 | 180 | 210 | 240 | 270 | 300 | 330 | 360 |
| Albumin (Control) | Δ | X | X | X | X | X | X | X | X | X | X | X |
| Albumin + Oleic acid | ◯ | ◯ | ◯ | ◯ | ◯ | ◯ | ◯ | ◯ | ◯ | ◯ | Δ | X |

Albumin = fatty acid-free human serum albumin (50 mg/mL)
Mixing of albumin and oleic acid at a molar ratio of 1:1
Checking of precipitation after quick spin every 30 min
Continuous heating until precipitation is formed in the mixture of albumin and oleic acid
After thermal treatment of 60 min, the control begins to become cloudy and is observed with the naked eye on dark background
After 180 min, the control becomes almost gelating
After 330 min, albumin mixed with oleic acid begins to be slightly clouded
Δ: slightly cloudy state, the cloudy state becomes weak when gently shaken
X: formation of precipitation

TABLE 4
70° C. Thermal treatment Conditions

| | Min | | | | | | | |
|---|---|---|---|---|---|---|---|---|
| Protein | 5 | 10 | 15 | 20 | 25 | 30 | 35 | 40 |
| Albumin (Control) | ◯ | Δ | X | X | X | X | X | X |
| Albumin + Oleic acid | ◯ | ◯ | ◯ | ◯ | ◯ | Δ | X | X |

Albumin = fatty acid-free human serum albumin (50 mg/mL)
Mixing of albumin and oleic acid at a molar ratio of 1:1
Checking of precipitation after quick spin every 5 min
Continuous heating until precipitation is formed in the mixture of albumin and oleic acid
After thermal treatment of 10 min, the control begins to become cloudy
After 15 min, the control is distinctly clouded
After 25 min, the control becomes completely gelating
After 30 min, albumin mixed with oleic acid begins to be slightly clouded
After 40 min, albumin mixed with oleic acid is distinctly clouded
Δ: slightly cloudy state, the cloudy state becomes weak when gently shaken
X: formation of precipitation

TABLE 5
75° C. Thermal treatment Conditions

| | Min | | |
|---|---|---|---|
| Protein | 3 | 6 | 9 |
| Albumin (Control) | ◯ | Δ | X |
| Albumin + Oleic acid | ◯ | Δ | X |

Albumin = fatty acid-free human serum albumin (50 mg/mL)
Mixing of albumin and oleic acid at a molar ratio of 1:1
Checking of precipitation after quick spin every 3 min
Continuous heating until precipitation is formed in the mixture of albumin and oleic acid
Similar patterns of precipitation in the control and the test group because of high temperature
After thermal treatment of 6 min, the control and albumin mixed with oleic acid begin to become cloudy
After 9 min, the control and albumin mixed with oleic acid are distinctly clouded
Δ: slightly cloudy state, the cloudy state becomes weak when gently shaken
X: formation of precipitation

TABLE 6

| Temp. | 65° C. | log65° C. | 70° C. | log70° C. | 75° C. | log75° C. |
|---|---|---|---|---|---|---|
| Albumin (Control) | 60 min | 1.78 | 15 min | 1.18 | 9 min | 0.95 |
| Albumin + Oleic acid | 360 min | 2.56 | 35 min | 1.54 | 9 min | 0.95 |

6.2. Effects of Various C18 Fatty Acids on Thermal Denaturation of Human Serum Albumin

Thermostabilizing effects were evaluated at 70° C. using other C18 fatty acids including stearic acid, linoleic acid, α-linolenic acid and γ-linolenic acid, as well as oleic acid, in the same manner as in Example 6.1. for testing of stability against denaturation due to thermal treatment using oleic acid. These effects were observed at an interval of 3 min up to 30 min, and then at an interval of 5 min. As a control, a fatty acid-free human serum albumin solution not processed with fatty acid as in Example 6.1. was used. As shown in Table 7 below, all of test groups processed with C18 fatty acids were confirmed to have superior stability against degeneration due to thermal treatment, compared to the human serum albumin control. As mentioned in Example 6.1, individual fatty acids were added at the same molar ratio as the albumin.

TABLE 7
70° C. Thermal treatment Conditions

| | Min | | | | | | | | | | | | | | | | |
|---|---|---|---|---|---|---|---|---|---|---|---|---|---|---|---|---|---|
| Protein | 3 | 6 | 9 | 12 | 15 | 18 | 21 | 24 | 27 | 30 | 35 | 40 | 45 | 50 | 55 | 60 | 65 |
| Albumin (Control) | ◯ | Δ | X | X | X | — | — | — | — | — | — | — | — | — | — | — | — |
| Albumin + Oleic acid | ◯ | ◯ | ◯ | ◯ | ◯ | ◯ | ◯ | ◯ | ◯ | ◯ | ◯ | ◯ | ◯ | Δ | Δ | Δ | X |
| Albumin + α- | ◯ | ◯ | ◯ | ◯ | Δ | Δ | Δ | Δ | X | X | X | — | — | — | — | — | — |
| Linolenic acid | | | | | | | | | | | | | | | | | |
| Albumin + γ- | ◯ | ◯ | ◯ | ◯ | Δ | Δ | Δ | Δ | X | X | X | — | — | — | — | — | — |
| Linolenic acid | | | | | | | | | | | | | | | | | |
| Albumin + | ◯ | ◯ | ◯ | ◯ | ◯ | ◯ | Δ | Δ | Δ | Δ | X | X | X | — | — | — | — |
| Linoleic acid | | | | | | | | | | | | | | | | | |
| Albumin + | ◯ | ◯ | Δ | X | X | X | — | — | — | — | — | — | — | — | — | — | — |
| Stearic acid | | | | | | | | | | | | | | | | | |

Albumin = fatty acid-free human serum albumin (50 mg/mL)
Mixing of albumin and fatty acid at a molar ratio of 1:1
Continuous heating until precipitation is formed in all samples
Control: After thermal treatment of 6 min, slight cloudiness; after 9 min, cloudy state to the extent of being observed with the naked eye; after 20 min, gelating and not mixed even after tapping
Mixture of albumin and stearic acid: After thermal treatment of 9 min, slight cloudiness appears and then becomes severe; after 12 min, cloudy state to the extent of being observed with the naked eye; after 20 min, gelation and not mixed even after tapping
Mixture with α- and γ-linolenic acid: after 12 min, cloudiness appears and then becomes severe in both; after 27 min, cloudy state to the extent of being observed with the naked eye
Mixture with linoleic acid: after 12 min, slight cloudiness appears and is maintained for a while; after 21 min, cloudy state; after 35 min, cloudy state to the extent of being observed with the naked eye
Δ: slightly cloudy state, the cloudy state becomes weak when gently shaken
X: formation of precipitation

6.3. Effects of Various C16 Fatty Acids on Thermal Denaturation of Human Serum Albumin

Thermostabilizing effects at 65° C. were evaluated using C16 fatty acids including palmitic acid and palmitoleic acid, as well as oleic acid as the C18 fatty acid, in the same manner as in Examples 6.1. and 6.2. for testing of stability against degeneration due to thermal treatment using various C18 fatty acids including oleic acid. The samples were warmed to 65° C. and observed at an interval of 5 min. As a control, a fatty acid-free human serum albumin solution not processed with fatty acid as in Examples 6.1. and 6.2. was used. As shown in Table 8 below, all of test groups processed with C16 fatty acids were confirmed to have much higher stability against denaturation due to thermal treatment, compared to the human serum albumin control. As mentioned in Examples 6.1. and 6.2, individual fatty acids were added at the same molar ratio as the albumin.

TABLE 8

| | Min | | | | | | | | | | | |
|---|---|---|---|---|---|---|---|---|---|---|---|---|
| Protein | 5 | 10 | 15 | 20 | 25 | 30 | 35 | 40 | 45 | 50 | 55 | 60 |
| Albumin (Control) | ◯ | ◯ | Δ | Δ | X | X | X | X | X | X | X | X |
| Albumin + Palmitic | ◯ | ◯ | ◯ | ◯ | ◯ | ◯ | ◯ | Δ | Δ | X | X | X |
| acid | | | | | | | | | | | | |
| Albumin + Palmitoleic | ◯ | ◯ | ◯ | ◯ | ◯ | ◯ | ◯ | ◯ | ◯ | ◯ | ◯ | ◯ |
| acid | | | | | | | | | | | | |
| Albumin + Oleic acid | ◯ | ◯ | ◯ | ◯ | ◯ | ◯ | ◯ | ◯ | ◯ | ◯ | ◯ | ◯ |

| | Min | | | | | | | | | | | |
|---|---|---|---|---|---|---|---|---|---|---|---|---|
| Protein | 65 | 70 | 75 | 80 | 85 | 90 | 95 | 100 | 105 | 110 | 115 | 120 |
| Albumin (Control) | X | X | X | X | X | X | X | X | X | X | X | X |
| Albumin + Palmitic | X | X | X | X | X | X | X | X | X | X | X | X |
| acid | | | | | | | | | | | | |
| Albumin + Palmitoleic | ◯ | ◯ | ◯ | Δ | Δ | Δ | Δ | Δ | Δ | Δ | Δ | Δ |
| acid | | | | | | | | | | | | |
| Albumin + Oleic acid | ◯ | ◯ | ◯ | ◯ | ◯ | ◯ | ◯ | ◯ | ◯ | ◯ | ◯ | ◯ |

| | Min | | | | | | | | | | | |
|---|---|---|---|---|---|---|---|---|---|---|---|---|
| Protein | 125 | 130 | 135 | 140 | 145 | 150 | 155 | 160 | 165 | 170 | 175 | 180 |
| Albumin (Control) | X | X | X | X | X | X | X | X | X | X | X | X |
| Albumin + Palmitic | X | X | X | X | X | X | X | X | X | X | X | X |
| acid | | | | | | | | | | | | |
| Albumin + Palmitoleic | Δ | X | X | X | X | X | X | X | X | X | X | X |
| acid | | | | | | | | | | | | |
| Albumin + Oleic acid | ◯ | ◯ | ◯ | ◯ | ◯ | ◯ | ◯ | ◯ | ◯ | ◯ | ◯ | ◯ |

Albumin = fatty acid-free human serum albumin (50 mg/mL),
Mixing of albumin and fatty acid at a molar ratio of 1:1
Checking of precipitation after quick spin every 5 min
Δ: slightly cloudy state, the cloudy state becomes weak when gently shaken
X: formation of precipitation

6.4. Effects of EPA and DEA on Thermal Denaturation of Human Serum Albumin

Thermostabilizing effects at 65° C. were evaluated using EPA (eicosapentaenoic acid) and DHA (docosahexanoic acid) as C20 and C22 fatty acids, as well as oleic acid as the C18 fatty acid, in the same manner as in Examples 6.1. to 6.3. for testing of stability against denaturation due to thermal treatment using various C16 and C18 fatty acids. The samples were warmed to 65° C. and observed at an interval of 5 min for initial 60 min, and at an interval of 10 min up to 130 min, and then at an interval of 30 min. As a control, a fatty acid-free human serum albumin solution not processed with fatty acid as in Examples 6.1. to 6.3. was used. As shown in Table 9 below, in all of test groups processed with EPA and DHA, stability against denaturation due to thermal treatment was much higher compared to the human serum albumin control and was similar to when using oleic acid. As mentioned in Examples 6.1. to 6.3, individual fatty acids were added at the same molar ratio as the albumin.

TABLE 9

| | Min | | | | | | | | | | | |
|---|---|---|---|---|---|---|---|---|---|---|---|---|
| Protein | 5 | 10 | 15 | 20 | 25 | 30 | 35 | 40 | 45 | 50 | 55 | 60 |
| Albumin (Control) | ◯ | ◯ | Δ | Δ | Δ | X | X | X | X | X | X | X |
| Albumin + EPA | ◯ | ◯ | ◯ | ◯ | ◯ | ◯ | ◯ | ◯ | ◯ | ◯ | ◯ | ◯ |
| Albumin + DHA | ◯ | ◯ | ◯ | ◯ | ◯ | ◯ | ◯ | ◯ | ◯ | ◯ | ◯ | ◯ |
| Albumin + Oleic acid | ◯ | ◯ | ◯ | ◯ | ◯ | ◯ | ◯ | ◯ | ◯ | ◯ | ◯ | ◯ |

| | Min | | | | | | | | | | | |
|---|---|---|---|---|---|---|---|---|---|---|---|---|
| Protein | 70 | 80 | 90 | 100 | 110 | 120 | 130 | 160 | 190 | 220 | 250 | 280 |
| Albumin (Control) | X | X | X | X | X | X | X | X | X | X | X | X |
| Albumin + EPA | ◯ | ◯ | ◯ | ◯ | ◯ | ◯ | ◯ | ◯ | ◯ | Δ | Δ | Δ |
| Albumin + DHA | ◯ | ◯ | ◯ | ◯ | ◯ | ◯ | ◯ | ◯ | ◯ | Δ | Δ | Δ |
| Albumin + Oleic acid | ◯ | ◯ | ◯ | ◯ | ◯ | ◯ | ◯ | ◯ | ◯ | Δ | Δ | Δ |

Albumin = fatty acid-free human serum albumin (50 mg/mL)
Mixing of albumin and fatty acid at a molar ratio of 1:1
Checking of precipitation after quick spin at predetermined time (at intervals of 5 min, 10 min or 30 min)
Δ: slightly cloudy state, the cloudy state becomes weak when gently shaken
X: formation of precipitation

### Example 7

### Testing for Structural Stability Against Thermal Treatment of Low-Concentration Human Serum Albumin Sample

(1) Preparation of Human Serum Albumin Sample

Each of Green Cross albumin, A3782 albumin (Sigma) and A1653 albumin (Sigma) was diluted with a 20 mM potassium phosphate buffer (pH 7.5) so as to have a concentration of 1 mg/mL. Then, oleic acid was added to each of Green Cross albumin and A3782 albumin so that the molar ratio of oleic acid and albumin was 1:1, after which reaction was carried out overnight at 4° C., thus preparing a total of five human serum albumin samples; i) Green Cross (room temperature), ii) A1653 albumin, iii) A3782 albumin, iv) A3782 albumin+oleic acid (1:1 molar ratio), v) Green Cross albumin+oleic acid (1:1 molar ratio).

(2) Measurement of Circular Dichroism of Albumin

The composition of the secondary structure of protein may be estimated using circular dichroism, and thus changes in structure of the thermally denatured protein may be measured. All of the original Green Cross albumin which was not thermally denatured and five albumin samples which were thermally denatured were diluted with a 20 mM potassium phosphate buffer (pH 7.5) so as to have a concentration of 0.25 mg/mL.

Measurement of circular dichroism was performed three times under conditions of a data pitch of 0.2 nm, a band width of 1.0 nm, a response time of 4 sec, and a scanning speed of 50 nm/min using a quartz cuvette having a path length of 0.1 cm by use of Jasco J-715 spectropolarimeter. The composition of the secondary structure was assayed from the measured circular dichroism values using SELCON3 program contained in the Dicroprot program.

(3) Testing for Structural Stability Against Thermal Treatment of Human Serum Albumin at 60° C.

Five human serum albumin samples (Green Cross albumin, A3782 albumin, A1653 albumin, Green Cross albumin+oleic acid (1:1 molar), A3782 albumin+oleic acid (1:1 molar ratio)) prepared in Example 7(1) were heated at 60° C. for 10 hr.

Then, circular dichroism thereof was measured in the manner as in Example 7(2). The resulting compositions of the secondary structure of the albumin samples are given in Table 10 below.

TABLE 10

| | | | | | Unor- |
|---|---|---|---|---|---|
| | α-Helix | β-sheet | Turn | poly(Pro)II | dered |
| Green Cross | 0.706 | 0.038 | 0.070 | 0.022 | 0.193 |
| (Room Temp.) | | | | | |
| A1653 Albumin | 0.481 | 0.072 | 0.105 | 0.042 | 0.311 |
| A3782 Albumin | 0.308 | 0.130 | 0.133 | 0.052 | 0.379 |
| Green Cross Albumin | 0.514 | 0.071 | 0.109 | 0.029 | 0.302 |
| A3782 Albumin + | 0.514 | 0.079 | 0.104 | 0.030 | 0.283 |
| Oleic acid (1:1) | | | | | |
| Green Cross Albumin + | 0.740 | 0.011 | 0.096 | 0.013 | 0.151 |
| Oleic acid (1:1) | | | | | |

As is apparent from Table 10, compared to the Green Cross (room temperature) sample which was not thermally denatured, the ratio of α-helix of the thermally denatured samples was generally decreased and the unordered structure thereof was increased.

However, the Green Cross albumin+oleic acid (1:1) sample had the ratio similar to that of the Green Cross (room temperature) sample and thus was very stable even when heated at 60° C. for 10 hr. Also, the A3782 albumin+oleic acid (1:1) sample had a higher ratio of α-helix even after thermal denaturation, compared to the A3782 albumin, thus exhibiting superior thermal stability. This indicates that thermal stability is increased when albumin is reacted with oleic acid.

(4) Testing for Structural Stability Against Thermal Treatment of Human Serum Albumin at 70° C.

Five human serum albumin samples (Green Cross albumin, A3782 albumin, A1653 albumin, Green Cross albumin+oleic acid (1:1 molar), A3782 albumin+oleic acid (1:1 molar ratio)) prepared in Example 7(1) were heated at 70° C. for 10 hr.

Then, circular dichroism thereof was measured in the manner as in Example 7(2). The resulting compositions of the secondary structure of the albumin samples are given in Table 11 below.

TABLE 11

| | | | | | Unor- |
|---|---|---|---|---|---|
| | α-Helix | β-sheet | Turn | poly(Pro)II | dered |
| Green Cross | 0.706 | 0.038 | 0.070 | 0.022 | 0.193 |
| (Room Temp.) | | | | | |
| A1653 Albumin | 0.330 | 0.124 | 0.144 | 0.055 | 0.369 |
| A3782 Albumin | 0.309 | 0.107 | 0.141 | 0.053 | 0.378 |
| Green Cross Albumin | 0.441 | 0.022 | 0.207 | 0.022 | 0.314 |
| A3782 Albumin + | 0.724 | 0.017 | 0.079 | 0.030 | 0.163 |
| Oleic acid (1:1) | | | | | |
| Green Cross Albumin + | 0.627 | 0.036 | 0.081 | 0.030 | 0.234 |
| Oleic acid (1:1) | | | | | |

As is apparent from Table 11, as shown by results of thermal treatment above, compared to the Green Cross (room temperature) sample which was not thermally denatured, the ratio of α-helix of the thermally denatured samples was generally decreased, and the unordered structure thereof was increased.

However, the Green Cross albumin+oleic acid (1:1 molar ratio) sample and the A3782 albumin+oleic acid (1:1 molar ratio) sample resulting from reaction with oleic acid and then thermal treatment had a ratio of α-helix similar to that of the Green Cross (room temperature) sample, and thus were very stable even when heated at 70° C. for 10 hr, compared to the albumins not reacted with oleic acid.

(5) Testing for Structural Stability Against Thermal Treatment of Human Serum Albumin at 80° C.

Five human serum albumin samples (Green Cross albumin, A3782 albumin, A1653 albumin, Green Cross albumin+oleic acid (1:1 molar), A3782 albumin+oleic acid (1:1 molar ratio)) prepared in Example 7(1) were heated at 80° C. for 10 hr.

Then, circular dichroism thereof was measured in the manner as in Example 7(2). The resulting compositions of secondary structure of the albumin samples are given in Table 12 below.

TABLE 12

| | | | | | Unor- |
|---|---|---|---|---|---|
| | α-Helix | β-sheet | Turn | poly(Pro)II | dered |
| Green Cross | 0.706 | 0.038 | 0.070 | 0.022 | 0.193 |
| (Room Temp.) | | | | | |
| A1653 Albumin | 0.270 | 0.127 | 0.149 | 0.054 | 0.390 |
| A3782 Albumin | 0.217 | 0.143 | 0.178 | 0.043 | 0.432 |
| Green Cross Albumin | 0.298 | 0.135 | 0.131 | 0.064 | 0.380 |
| A3782 Albumin + | 0.212 | 0.169 | 0.162 | 0.066 | 0.404 |
| Oleic acid (1:1) | | | | | |
| Green Cross Albumin + | 0.132 | 0.223 | 0.188 | 0.061 | 0.404 |
| Oleic acid (1:1) | | | | | |

As is apparent from Table 12, as shown by results of thermal treatment above, compared to the Green Cross (room temperature) sample which was not thermally denatured, the ratio of α-helix of all of the thermally denatured samples was decreased, and the unordered structure thereof was increased.

Unlike at 60° C. and 70° C., for both of the Green Cross albumin+oleic acid (1:1 molar ratio) sample and the A3782 albumin+oleic acid (1:1 molar ratio) sample, the ratio of α-helix decreased, and the unordered structure was increased.

### Example 8

### Testing for Concentration Range of Albumin Maintaining Thermal Stability

A3782 albumin (Sigma) was diluted with a 20 mM potassium phosphate buffer (pH 7.5) so that its concentration was 1 mg/mL, 5 mg/mL, 10 mg/mL, 15 mg/mL, 20 mg/mL, 25 mg/mL, 30 mg/mL, 35 mg/mL, 40 mg/mL and 45 mg/mL. Then, oleic acid was added to the A3782 albumin at a molar ratio of 1:1 and then reacted overnight at 4° C.

Subsequently, ten samples including 1 mg/mL A3782 albumin+oleic acid (1:1 molar ratio), 5 mg/mL A3782 albumin+oleic acid (1:1 molar ratio), 10 mg/mL A3782 albumin+oleic acid (1:1 molar ratio), 15 mg/mL A3782 albumin+oleic acid (1:1 molar ratio), 20 mg/mL A3782 albumin+oleic acid (1:1 molar ratio), 25 mg/mL A3782 albumin+oleic acid (1:1 molar ratio), 30 mg/mL A3782 albumin+oleic acid (1:1 molar ratio), 35 mg/mL A3782 albumin+oleic acid (1:1 molar ratio), 40 mg/mL A3782 albumin+oleic acid (1:1 molar ratio), and 45 mg/mL A3782 albumin+oleic acid (1:1 molar ratio) were heated at 70° C. for 10 hr.

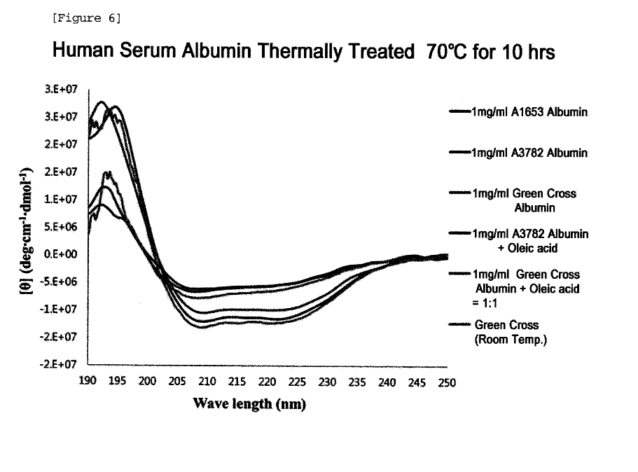

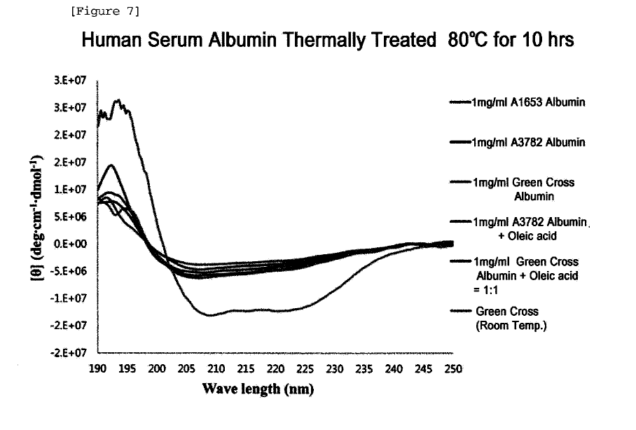

Circular dichroism of the albumins was measured in the manner as in Example 7(2). As shown by results, the 1 mg/mL A3782 albumin+oleic acid (1:1 molar ratio) sample had the ratio of α-helix similar to that of the Green Cross sample which was not thermally denatured (Table 11 and FIG. 6). In the circular dichroism graph of the thermally denatured 1 to 45 mg/mL A3782 albumin+oleic acid (1:1 molar ratio) samples, the 1 to 40 mg/mL A3782 albumin+oleic acid (1:1 molar ratio) samples had similar spectrum to that of the 1 mg/mL A3782 albumin+oleic acid (1:1 molar ratio) sample, and thereby it was confirmed that the composition of the secondary structure of protein was not greatly changed (FIG. 7).

Whereas, the 45 mg/mL A3782 albumin+oleic acid (1:1) sample had a remarkably weakened circular dichroism signal and thus the ratio of α-helix was decreased, and the unordered structure was increased. Thereby, the A3782 albumin+oleic acid (1:1 molar ratio) sample was confirmed to be maintained in high thermal stability in the concentration range of 1 to 40 mg/mL (FIG. 8).

### Example 9

### Evaluation of Thermal Stability of Albumin Depending on Addition of C16 to C22 Unsaturated Fatty Acid

A3782 albumin (Sigma) was diluted with a 20 mM potassium phosphate (pH 7.5) so as to have a concentration of 1 mg/mL. Then, oleic acid, linoleic acid, α-linolenic acid, γ-linolenic acid and palmitoleic acid were added to the A3782 albumin at a molar ratio of 1:1 relative to the albumin, and then reacted overnight at 4° C.

Five samples, including A3782 albumin+oleic acid (1:1 molar ratio), A3782 albumin+linoleic acid (1:1 molar ratio), A3782 albumin+α-linolenic acid (1:1 molar ratio), A3782 albumin+γ-linolenic acid (1:1 molar ratio), A3782 albumin+palmitoleic acid (1:1 molar ratio), were heated at 70° C. for 10 hr.

Circular dichroism of the albumins was measured in the same manner as in Example 7(2). As shown by results, the A3782 albumin+oleic acid (1:1 molar ratio) sample had the ratio of α-helix similar to that of the Green Cross sample which was not thermally denatured (Table 11 and FIG. 6). In the circular dichroism graph of the thermally denatured albumin samples added with C16 to C24 unsaturated fatty acids, the samples added with C16 to C22 unsaturated fatty acids other than oleic acid were generally similar in circular dichroism spectrum to that of the sample added with oleic acid, and thereby it was confirmed that there was no great change in the secondary structure of protein (FIG. 9).

This indicates that the same or corresponding effects may be obtained even when the C16 to C22 fatty acid in addition to oleic acid is applied to the method of the present invention.

Although the embodiments of the present invention have been disclosed for illustrative purposes, those skilled in the art will appreciate that a variety of different modifications, additions and substitutions are possible, without departing from the scope and spirit of the invention as disclosed in the accompanying claims. Accordingly, such modifications, additions and substitutions should also be understood as falling within the scope of the present invention.

1. A composition for thermostabilization in the preparation of a human serum albumin formulation, comprising a fatty acid selected from the group consisting of C16 to C22 fatty acids or a pharmaceutically acceptable salt thereof.

2. The composition of claim 1, wherein the human serum albumin is human plasma-derived or recombinant human serum albumin.

3. The composition of claim 1, wherein the fatty acid is selected from the group consisting of palmitic acid, palmitoleic acid, stearic acid (octadecanoic acid), oleic acid, linoleic acid, linolenic acid, EPA (eicosapentaenoic acid) and DHA (docosahexaenoic acid).

4. The composition of claim 1, wherein the salt of the fatty acid is an alkali metal or alkaline earth metal salt.

5. The composition of claim 1, wherein the fatty acid is added at a molar ratio of 1:0.5 to 1:2 relative to the albumin.

6. A method of preparing thermally stabilized human serum albumin, comprising:
1) adjusting a concentration of human serum albumin to 1 to 40 mg/mL; and
2) adding a fatty acid selected from the group consisting of C16 to C22 fatty acids or a pharmaceutically acceptable salt thereof to the human serum albumin.

7. The method of claim 6, wherein steps 1) and 2) are performed sequentially or reversely.

8. The method of claim 6, wherein the fatty acid is one or more fatty acids selected from the group consisting of palmitic acid, palmitoleic acid, stearic acid (octadecanoic acid), oleic acid, linoleic acid, linolenic acid, EPA (eicosapentaenoic acid) and DHA (docosahexaenoic acid).

9. The method of claim 6, wherein when the thermally stabilized human serum albumin is thermally treated at 60 to 80° C. for 6 to 15 hr, 80% or more of α-helix thereof is maintained.

10. The method of claim 6, wherein the fatty acid or pharmaceutically acceptable salt thereof is added at a molar ratio of 1:0.5 to 1:2 relative to the human serum albumin.

11. A method of preparing a human serum albumin formulation, comprising adding a fatty acid selected from the group consisting of C16 to C22 fatty acids or a pharmaceutically acceptable salt thereof to human serum albumin and thermally treating the resultant.

12. The method of claim 11, wherein the human serum albumin is human plasma-derived or recombinant human serum albumin.

13. The method of claim 11, wherein the human serum albumin is prepared by a method comprising culturing a transformed cell which expresses human serum albumin having an amino acid sequence of SEQ ID NO: 1.

14. The method of claim 11, wherein the fatty acid selected from the group consisting of C16 to C22 fatty acids is added to an isolated human serum albumin.

15. The method of claim 14, wherein the isolated human serum albumin is obtained from blood or serum of a transgenic animal or a transformed cell which expresses human serum albumin; from a culture or culture broth of the cell; or from blood or serum of human.

16. The method of claim 11, wherein a concentration of the human serum albumin is adjusted to 1 to 40 mg/mL before or after adding the fatty acid selected from the group consisting of C16 to C22 fatty acids or pharmaceutically acceptable salt thereof.

17. The method of claim 16, wherein the thermal treatment is performed at 60 to 80° C.

18. The method of claim 17, further comprising concentrating the thermally treated human serum albumin to a concentration of 50 to 200 mg/mL.
